SEQUENCE LISTING (15 sequences)

SEQ ID NO: 1 - protein, 24 aa, Artificial Sequence
  Synthetic peptide
VLGGGXALLRXIPALDSLXPANED

SEQ ID NO: 2 - protein, 24 aa, Artificial Sequence
  Synthetic peptide
VLGGGVALLRVIPALDSLTPANED

SEQ ID NO: 3 - protein, 20 aa, Artificial Sequence
  Synthetic peptide
KFGADARALMLQGVDLLADA

SEQ ID NO: 4 - protein, 20 aa, Artificial Sequence
  Synthetic peptide
NPVEIRRGVMLAVDAVIAEL

SEQ ID NO: 5 - protein, 20 aa, Artificial Sequence
  Synthetic peptide
VIAELKKQSKPVTTPEEIAQ

SEQ ID NO: 6 - protein, 20 aa, Artificial Sequence
  Synthetic peptide
EEIAQVATISANGDKEIGNI

SEQ ID NO: 7 - protein, 20 aa, Artificial Sequence
  Synthetic peptide
RKGVITVKDGKTLNDELEII

SEQ ID NO: 8 - protein, 20 aa, Artificial Sequence
  Synthetic peptide
QSIVPALEIANAHRKPLVII

SEQ ID NO: 9 - protein, 20 aa, Artificial Sequence
  Synthetic peptide
LVLNRLKVGLQVVAVKAPGF

SEQ ID NO: 10 - protein, 20 aa, Artificial Sequence
  Synthetic peptide
GEVIVTKDDAMLLKGKGDKA

SEQ ID NO: 11 - protein, 20 aa, Artificial Sequence
  Synthetic peptide
VTDALNATRAAVEEGIVLGG

SEQ ID NO: 12 - protein, 20 aa, Artificial Sequence
  Synthetic peptide
IVLGGGCALLRCIPALDSLT

SEQ ID NO: 13 - protein, 20 aa, Artificial Sequence
  Synthetic peptide
EIIKRTLKIPAMTIAKNAGV

SEQ ID NO: 14 - protein, 20 aa, Artificial Sequence
  Synthetic peptide
VNMVEKGIIDPTKVVRTALL

SEQ ID NO: 15 - protein, 14 aa, Artificial Sequence
  Synthetic peptide
GKVGEVIVTKDDAM

(19) United States
(12) Patent Application Publication
(10) Pub. No.: US 2015/0018271 A1
(43) Pub. Date: Jan. 15, 2015

(54) HSP60 DERIVED PEPTIDES AND PEPTIDE ANALOGS FOR SUPPRESSION AND TREATMENT OF NON-AUTOIMMUNE DIABETES

(71) Applicant: Yeda Research and Development Co. Ltd., Rehovot (IL)

(72) Inventors: Irun R. Cohen, Rehovot (IL); Raanan Margalit, Rehovot (IL)

(21) Appl. No.: 14/379,758

(22) PCT Filed: Feb. 28, 2013
(86) PCT No.: PCT/IL2013/050174

§ 371 Date: Aug. 20, 2014

Related U.S. Application Data
(60) Provisional application No. 61/605,262, filed on Mar. 1, 2012.

(51) Int. Cl. A61K 38/17; A61K 45/06

(52) U.S. Cl. CPC A61K 38/17; A61K 45/06
USPC 514/6.9

(57) ABSTRACT

The present invention provides methods of prevention and treatment of Type 2 diabetes (T2D) using peptides and analogs of heat shock protein 60 (hsp60), and for suppression, prevention and treatment of complications associated with T2D. The invention is exemplified using DiaPep277™, a peptide analog of human hsp60. The invention further relates treatment regimens useful for suppression, prevention or treatment of T2D.

### FIELD OF THE INVENTION

The invention relates to methods for prevention, suppression and treatment of non-autoimmune diabetes, comprising administration of a peptide derived from heat shock protein 60 (hsp60), or an analog thereof. The invention is exemplified by use of the hsp60 peptide analog denoted DiaPep277-DiaPep277 for treatment of Type 2 diabetes (T2D). The present invention further relates to treatment regimens and formulations adapted for administration of DiaPep277 and other hsp60 peptides and analogs for suppression or treatment of T2D.

### BACKGROUND OF THE INVENTION

Type 2 Diabetes (T2D, also referred to as non-insulin-dependent diabetes mellitus, NIDDM, or adult-onset diabetes) is the most common form of diabetes, accounting for 90% of cases of diabetes. It is a metabolic disorder that is characterized by high blood glucose in the context of relative peripheral insulin resistance and insulin deficiency. The occurrence of diabetes in persons 45 to 64 years of age is 7 percent, but the incidence increases significantly in persons 65 years of age or older. Type 1 diabetes (T1D, also referred to as insulin dependent diabetes mellitus, IDDM), which accounts for about 10% of the diabetic cases is an autoimmune disease that results from the destruction of the beta-cells in the pancreas.

Obesity or metabolic syndrome is the primary cause of T2D in people who are genetically predisposed to the disease. Type 2 diabetes is initially managed by increasing exercise and dietary modification. If blood sugars are not lowered by these measures, medications such as metformin or insulin may be needed.

Rates of diabetes have increased markedly over the last 50 years in parallel with obesity. As of 2010 there were approximately 285 million people with the disease compared to around 30 million in 1985. Type 2 diabetes is typically a chronic disease, associated with a ten year shorter life expectancy. This is partly due to a number of complications with which it is associated including: two to four times the risk of cardiovascular disease and stroke, a 20-fold increase in lower limb amputations, and increased rates of hospitalizations. In the developed world, and increasingly elsewhere, T2D is the largest cause of non-traumatic blindness and kidney failure. It has been associated with an increased risk of cognitive dysfunction and dementia through disease processes such as Alzheimer's disease and vascular dementia. Other complications include: acanthosis nigricans, sexual dysfunction, and frequent infections.

Heat shock proteins (HSPs) are highly conserved proteins expressed in all prokaryotic and eukaryotic cells. They are involved in many important cellular processes such as correct folding of newly synthesized proteins and subunit assembly and therefore are termed molecular chaperones (Bukau, B., et al. 2000, Cell 101, 119-122). Under non-physiological conditions like high temperature, ultraviolet radiation, viral or bacterial infection, cellular HSP expression is up-regulated. HSPs exert cytoprotective functions such as preventing the aggregation of denatured proteins, initiating their refolding or proteolytic degradation. According to their molecular weight, HSPs are divided into six subfamilies: small HSPs, HSP40, HSP60, HSP70, HSP90 and HSP100. They are located in the cytosol (HSP70, HSP90, HSP100), in the endoplasmic reticulum (HSP70, HSP90) or in mitochondria (HSP60).

The HSP60, HSP70, and HSP90 subfamilies have attracted increasing attention because of their potential roles in immunologically relevant processes. Several studies have identified HSPs as targets of immune responses during microbial infections (Zugel, U., and Kaufmann, S. H., 1999, Immunobiology 201, 22-35). Because of the high sequence homology between microbial HSPs and endogenous HSPs derived from damaged or stressed tissue, immunological cross-reactivity was suggested to contribute to the development of autoimmune disorders including rheumatoid arthritis and diabetes (Holoshitz, J., et al. 1986, Lancet 2, 305-309; Elias, D., et al., 1991, Proc. Natl. Acad. Sci. U.S.A 88, 3088-3091; Abulafia-Lapid, R., et al., 1999, J. Autoimmun. 12, 121-129).

Many publications disclose uses of heat shock proteins or fragments thereof as immune modulators in diagnosis, treatment or prevention of autoimmune diseases. Most of these disclosures relate to hsp60, or fragments of this protein. Antibodies against human hsp60, which has a high homology to bacterial hsp65, have been found in the circulation at the onset of T1D in humans and in pre-diabetic NOD-mice. Elias et al. (Diabetes 1997, 46, 758-64) demonstrated a specific peptide of human hsp60, designated p277, to be one of the immunodominant epitopes in autoimmune diabetes. Accordingly, T-cell reactivity to p277 has been reported at the onset of diabetes in NOD mice. Subcutaneous administration of p277 down-regulated T-cell reactivity to beta cell antigens and prevented the development of diabetes in NOD mice. Treatment induced p277-specific IgG1 antibodies as well as an increase in p277-specific IL-4 and IL-10 secretion and a decrease in gamma interferon secretion, suggesting an up-regulation of the Th2 cytokine pathway. As destruction of the islets of Langerhans in the pancreas is believed to be a Th1 response, a shift of Th1 to Th2 response induced by p277 could be the cause of the attenuation of T1D.

U.S. Pat. Nos. 5,114,844; 5,671,848; 5,578,303 and 5,780,034 disclose the use of hsp60 in diagnosis and treatment of T1D. It has been further disclosed (U.S. Pat. Nos. 6,180,103 and 5,993,803 and WO 96/19236, WO 97/01959 and WO 98/08536) that fragments and peptide analogs of this hsp60 protein may serve as therapeutically useful entities in preventing or alleviating T1D and host vs. graft disease.

A peptide analog of human hsp60 p277, denoted herein DiaPep277, disclosed in U.S. Pat. No. 6,180,103 and WO 96/19236 as p277(Val⁶, Val¹¹) is a synthetic analog in which the two native cysteine residues at positions 6 and 11 are replaced with Valine residues.

WO 03/070761 discloses anti-inflammatory hsp60 derived peptides including a minimal epitope of the peptide p277 that are capable of reacting via the Toll like receptor 2 (T1r2) on T cells, without necessarily activating the TCR of these cells.

WO 2005/072056 discloses the use of DiaPep277, in conjunction with low-antigenicity diet, for delaying the onset of autoimmune diseases, particularly Type 1 diabetes, and to methods useful for prevention, delay, suppression or treatment of autoimmune diseases using oral administration of DiaPep277.

WO 2006/072946 discloses the use of p277 and its analogs in modulation of immune responses and inflammatory diseases and specifically in the treatment or prevention of hepatic disorders.

DiaPep277 has successfully completed a phase III clinical trial for the treatment of subjects with new onset T1D. The trial results show that DiaPep277 preserves the residual beta cells in T1D by modulating and arresting the autoimmune destruction of beta cells.

DiaPep277 has not been taught or suggested to be capable of treating T2D, which is a disease associated with resistance to insulin in the tissues of the body (liver, muscle, adipose, etc) and is not associated with an autoimmune etiology.

There is an unmet need to provide effective and safe compositions for prevention, delay, suppression and treatment of non-autoimmune diabetes, namely T2D.

### SUMMARY OF THE INVENTION

The present invention provides pharmaceutical compositions comprising DiaPep277 (SEQ ID NO: 2) and other hsp60 derived peptides and peptide analogs, formulations and methods of suppression and treatment of non-autoimmune diabetes, and uses thereof for prevention and alleviation of complications associated with Type 2 diabetes (T2D).

It was unexpectedly found that DiaPep277 which is effective in treatment of T1D by modulating and arresting the autoimmune destruction of beta cells is therapeutically beneficial also in T2D, a disease with no autoimmune etiology. It was further found that schedules used for treating T1D with DiaPep277 are ineffective in T2D and therefore new treatment schedules and formulations are herein provided. According to the present invention, a new diabetic patient population is provided as a target for the hsp60 peptide analog DiaPep277. Thus, the present invention discloses treatment of patients having T2D and no apparent autoimmune component and those who are genetically predisposed to the disease.

The present invention provides, according to one aspect, a method of treating or alleviating of T2D comprising administering to a patient in need thereof, a composition comprising a peptide derived from hsp60 or a peptide analog thereof.

According to some embodiments, the peptide analog consists of a sequence corresponding to amino acid residues 437-460 of human hsp60 having the sequence: Val-Leu-Gly-Gly-Gly-X₁-Ala-Leu-Leu-Arg-X₂-IIe-Pro-Ala-Leu-Asp-Ser-Leu-X₃-Pro-Ala-Asn-Glu-Asp (SEQ ID NO:1), wherein X1 is a Cys or Val residue, X₂ is a Cys or Val residue, and X₃ is a Thr or Lys residue.

According to particular embodiments, X₁ and X₂ are Val and X₃ is Thr and the peptide analog is 24-30 amino acids long.

According to particular embodiments, the peptide analog it is a Val⁶, Val¹¹ analog of residues 437-460 of hsp60, as set forth in SEQ ID NO:2:

(SEQ ID NO: 2)
1 6 11
Val-Leu-Gly-Gly-Gly-Val-Ala-Leu-Leu-Arg-Val-Ile-Pro-Ala-Leu-Asp-Ser-Leu-Thr-Pro-
24
Ala-Asn-Glu-Asp,

denoted herein DiaPep277.

According to another embodiment, the hsp60 fragment peptide is selected from the group consisting of:

residues 31-50 of human hsp60:
(SEQ ID NO: 3)
Lys-Phe-Gly-Ala-Asp-Ala-Arg-Ala-Leu-Met-Leu-Gln-
Gly-Val-Asp-Leu-Leu-Ala-Asp-Ala;
residues 136-155 of human hsp60:
(SEQ ID NO: 4)
Asn-Pro-Val-Glu-Ile-Arg-Arg-Gly-Val-Met-Leu-Ala-
Val-Asp-Ala-Val-Ile-Ala-Glu-Leu;
residues 151-170 of human hsp60:
(SEQ ID NO: 5)
Val-Ile-Ala-Glu-Leu-Lys-Lys-Gln-Ser-Lys-Pro-Val-
Thr-Thr-Pro-Glu-Glu-Ile-Ala-Gln;
residues 166-185 of human hsp60:
(SEQ ID NO: 6)
Glu-Glu-Ile-Ala-Gln-Val-Ala-Thr-Ile-Ser-Ala-Asn-
Gly-Asp-Lys-Glu-Ile-Gly-Asn-Ile;
residues 195-214 of human hsp60:
(SEQ ID NO: 7)
Arg-Lys-Gly-Val-Ile-Thr-Val-Lys-Asp-Gly-Lys-Thr-
Leu-Asn-Asp-Glu-Leu-Glu-Ile-Ile;
residues 255-274 of human hsp60:
(SEQ ID NO: 8)
Gln-Ser-Ile-Val-Pro-Ala-Leu-Glu-Ile-Ala-Asn-Ala-
His-Arg-Lys-Pro-Leu-Val-Ile-Ile;
residues 286-305 of human hsp60:
(SEQ ID NO: 9)
Leu-Val-Leu-Asn-Arg-Leu-Lys-Val-Gly-Leu-Gln-Val-
Val-Ala-Val-Lys-Ala-Pro-Gly-Phe;
residues 346-365 of human hsp60:
(SEQ ID NO: 10)
Gly-Glu-Val-Ile-Val-Thr-Lys-Asp-Asp-Ala-Met-Leu-
Leu-Lys-Gly-Lys-Gly-Asp-Lys-Ala;
residues 421-440 of human hsp60:
(SEQ ID NO: 11)
Val-Thr-Asp-Ala-Leu-Asn-Ala-Thr-Arg-Ala-Ala-Val-
Glu-Glu-Gly-Ile-Val-Leu-Gly-Gly;
residues 436-455 of human hsp60:
(SEQ ID NO: 12)
Ile-Val-Leu-Gly-Gly-Gly-Cys-Ala-Leu-Leu-Arg-Cys-
Ile-Pro-Ala-Leu-Asp-Ser-Leu-Thr;
residues 466-485 of human hsp60:
(SEQ ID NO: 13)
Glu-Ile-Ile-Lys-Arg-Thr-Leu-Lys-Ile-Pro-Ala-Met-
Thr-Ile-Ala-Lys-Asn-Ala-Gly-Val;
residues 511-530 of human hsp60:
(SEQ ID NO: 14)
Val-Asn-Met-Val-Glu-Lys-Gly-Ile-Ile-Asp-Pro-Thr-
Lys-Val-Val-Arg-Thr-Ala-Leu-Leu;
residues 343-366 of human hsp60:
(SEQ ID NO: 15)
Gly-Lys-Val-Gly-Glu-Val-Ile-Val-Thr-Lys-Asp-Asp-
Ala-Met.

The hsp60 peptide or peptide analog is administered, according to the invention, within a pharmaceutical composition comprising at least one pharmaceutically acceptable excipient, diluent, adjuvant or salt.

The pharmaceutical composition may be administered to a subject in need thereof, by any administration route, including but not limited to: intramuscular, intravenous, oral, intraperitoneal, subcutaneous, topical, intradermal or transdermal delivery.

According to some embodiments, the composition is administered by a route selected from the group consisting of: subcutaneous injection (SC), intra-peritoneal (IP) injection, intra-muscular (IM) injection and intra-venous (IV) injection. According to a particular embodiment, the composition is administered orally (PO).

According to yet other embodiment the pharmaceutical composition comprises hsp60-derived peptide or peptide analog in an aqueous solution, including but not limited to saline, PBS and water. Each possibility represents a separate embodiment of the present invention.

According to some embodiments, the composition comprises an adjuvant. Pharmaceutically acceptable adjuvants include, but are not limited to water in oil emulsion, lipid emulsion, or submicron oil in water emulsion and liposomes. According to specific embodiments the adjuvant is Intralipid® or Lipofundin®.

In some embodiments the composition is formulated for intramuscular, intravenous, oral, intraperitoneal, subcutaneous, topical, intradermal or transdermal delivery.

According to some embodiments, a weekly dose of at least 2 mg of the hsp60 derived peptide or peptide analog is provided. According to other embodiments, an individual dose comprising at least 5 mg of the hsp60 derived peptide or peptide analog is provided. According to specific embodiments, an individual dose comprising 10 mg of the hsp60 derived peptide or peptide analog is provided. According to particular embodiments, 2-50 mg of an hsp60 peptide or peptide analog is administered in 1-5 weekly doses. Each possibility represents a separate embodiment of the present invention.

According to some embodiments, the hsp60 derived peptide or peptide analog is administered to a subject in need thereof 1-24 times per month. According to yet other embodiments, the hsp60 derived peptide or peptide analog is administered to a subject in need thereof at least once a week. According to some particular embodiments, the hsp60 derived peptide or peptide analog is administered to a subject in need thereof 2-5 times per week. According to yet other embodiments, daily administration is provided. Each possibility represents a separate embodiment of the present invention.

According to some particular embodiments, a composition comprising 2-10 mg of the hsp60 derived peptide or peptide analog is administered to a subject in need thereof at least once a week by a route selected from the group consisting of: subcutaneous (SC) injection, intra-peritoneal (IP) injection, intra-muscular (IM) injection and intra-venous (IV) injection. According to particular embodiments, the pharmaceutical composition administered by subcutaneous injection comprises fat emulsion. Each possibility represents a separate embodiment of the present invention.

According to other embodiments, a composition comprising 50-500 mg hsp60 peptide or analog is provided for oral administration to a subject in need thereof in a schedule of 4-30 times per month. According to some embodiments, oral administration is at least once a week. According to other embodiments, oral administration is 2-5 times per week. According to yet other embodiments, oral administration is daily. Each possibility represents a separate embodiment of the present invention.

According to some embodiments, the hsp60 derived peptide or peptide analog is administered as part of a treatment regimen comprising administering to the patient insulin. According to yet other embodiments, the subject does not receive insulin as part of its treatment.

The present invention further provides a method of suppression, prevention or treatment of complications of T2D, comprising administering to a patient in need of such treatment a pharmaceutical composition comprising at least one peptide derived from hsp60 or an analog thereof.

According to a particular embodiment the peptide analog is DiaPep277.

T2D complications which may be prevented, suppressed or treated according to the present invention, include but are not limited to: metabolic syndrome, fatty liver, insulin resistance, cancer, microvascular complications including neuropathy (nerve damage), nephropathy (kidney disease) and vision disorders (e.g., retinopathy, glaucoma, cataract and corneal disease), macrovascular complications including heart disease, stroke and peripheral vascular disease (which can lead to ulcers, gangrene and amputation).

Other complications of diabetes include infections, metabolic difficulties, impotence, autonomic neuropathy and pregnancy problems.

According to some embodiments, the method further comprises administration of at least one additional anti-diabetic agent.

According to some embodiments the at least one additional anti-diabetic agent is selected from the group consisting of: insulin, sulfonylureas, alpha-glucosidase inhibitors, biguanides, meglitinides, and thiazolidinediones.

According to other embodiments the at least one additional anti-diabetic agent is selected from the group consisting of: Sensitizers (such as biguanides and thiazolidinediones); secretagogues (such as sulfonylureas and nonsulfonylurea secretagogues); alpha-glucosidase inhibitors; peptide analogs (such as injectable incretin mimetics and injectable Amylin analogues).

According to some particular embodiments, the anti-diabetic agent is selected from the group consisting of: Metformin; rosiglitazone (Avandia); pioglitazone (Actos); tolbutamide (Orinase); acetohexamide (Dymelor); tolazamide (Tolinase); chlorpropamide (Diabinese); Second-generation agents; glipizide (Glucotrol); glyburide (Diabeta, Micronase, Glynase); glimepiride (Amaryl); gliclazide (Diamicron); repaglinide (Prandin); nateglinide (Starlix); miglitol (Glyset); acarbose (Precose/Glucobay); Exenatide; Liraglutide; vildagliptin (Galvus); sitagliptin (Januvia); saxagliptin (Onglyza); linagliptin (Tradjenta).

According a particular embodiment, the hsp60 derived peptide or analog is administered to a subject in need thereof, as part of a treatment regimen which does not include administration of other anti-diabetic agents.

According to another aspect the present invention provides a method for delaying the onset of T2D in patients having no apparent autoimmune component and those who are genetically predisposed to the disease, comprising administering of an hsp60 derived peptide or peptide analog.

According to some particular embodiments, the hsp60 peptide analog is DiaPep277.

The present invention also discloses hsp60 derived peptides and peptide analogs for use in preventing, delaying the onset or treating T2D.

Also disclosed is the use of hsp60 derived peptide or analog for preparation of a medicament for prevention, delaying the onset or treatment of T2D.

According to some embodiments the hsp60 peptide analog is DiaPep277 for treatment of T2D.

Pharmaceutical compositions comprising an hsp60 peptide or analog, specifically formulated for use in prevention, delaying the onset or treatment of T2D are also within the scope of the present invention.

According to some embodiments, a pharmaceutical composition comprising an hsp60 peptide or analog is provided, further comprising another anti-diabetic agent.

According to particular embodiments, the pharmaceutical composition comprises the hsp60 peptide analog DiaPep277 in addition to another anti-diabetic agent.

The present invention provides according to another aspect long acting pharmaceutical compositions comprising DiaPep277 or a pharmaceutically acceptable salt thereof, specifically formulated for providing a therapeutically effective amount of the peptide over a period selected from 2-6 days, one week, two weeks or longer.

According to some embodiments, the long acting pharmaceutical composition is for use in treatment of T2D.

According to some specific embodiments, the long acting pharmaceutical composition is provided in depot form suitable for injection or implantation at a medically acceptable location in a subject in need thereof.

According to some embodiments, the long acting pharmaceutical composition is suitable for a dosing schedule from once weekly to once in every 6 months.

According to particular embodiments, the composition is suitable for a dosing schedule from once every 2 weeks to once monthly. Each possibility represents a separate embodiment of the present invention.

Specific examples of the long acting compositions include biodegradable or non-biodegradable microspheres, implants of any suitable geometric shape, implantable rods, implantable capsules, implantable rings, prolonged release gels and erodible matrices. Each possibility represents a separate embodiment of the invention.

The present invention further provides a method of treating T2D, comprising the administration or implantation of a composition comprising a therapeutically effective amount of a pharmaceutically acceptable salt of DiaPep277.

The long acting pharmaceutical compositions according to the principles of the present invention provide equal or superior therapeutic efficacy to the commercially available injectable dosage forms, with reduced incidence and/or severity of side effects at the local and/or systemic levels.

According to certain embodiments, the implantable depot is suitable for subcutaneous or intramuscular implantation.

According to alternative embodiments, the long acting parenteral pharmaceutical composition comprises a pharmaceutically acceptable biodegradable or non-biodegradable carrier for DiaPep277.

According to another aspect, the present invention provides a kit comprising DiaPep277 in a dose form of at least 2 mg formulated for administration to patients having T2D. According to other embodiments, the kit comprises 5 mg of DiaPe277. The kit may further comprise instructions for the administration of the composition.

### BRIEF DESCRIPTION OF THE FIGURES

FIG. 1 depicts mice weight over time following various schedules of DiaPep277 administration or controls.

FIG. 2 represents blood glucose levels over time following various schedules of DiaPep277 administration or controls.

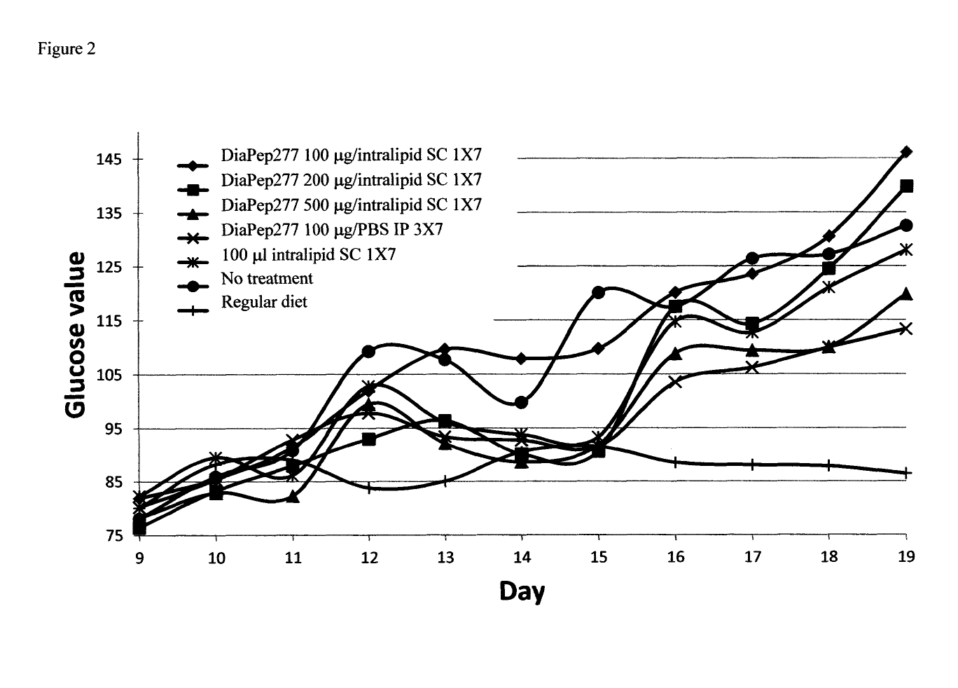

### DETAILED DESCRIPTION OF THE INVENTION

Hsp60 derived peptides and peptide analogs, especially the peptide analog DiaPep277, are known to be effective in treatment of T1D. It was now unexpectedly found that they are also effective in non-autoimmune diabetes, namely T2D. In order to be effective in T2D, the therapeutic agent needs to penetrate the adipose tissue beyond the lymphoid organs and affect the chronic inflammation in the tissue. Therefore, in T2D different doses and treatment schedules than those used in T1D must be used.

The effective treatment of T1D with DiaPep277 as known in the art is typically 1 mg administered every three months by subcutaneous injection.

It is now disclosed for the first time that a dose of at least 2 mg of DiaPep277 for T2D administered by subcutaneous injection, by intra-peritoneal (IP) injection, intra-muscular (IM) injection or intra-venous (IV) injection, is effective in reducing blood glucose levels in T2D animals.

Typically, for subcutaneous injection DiaPep277 is formulated with adjuvant, such as fat emulsion while the IP and IV routes of injection are without the adjuvant, but other formulations and schedules are possible according to the present invention. DiaPep277, typically without adjuvant, may be also administered orally (PO) at doses of 100 mg or greater. The frequency of administration is according to some embodiments of the present invention, between once a month and four times a month.

Pharmaceutical compositions comprising hsp60 derived peptides and analog are also disclosed in the present invention, together with novel formulations and treatment schedules, for use in prevention, suppression or treatment of T2D complications. Such complications include but are not limited to: metabolic syndrome, fatty liver, neuropathy, nephropathy, retinopathy, heart disease, peripheral vascular disease and cancer.

In addition, it is herein disclosed for the first time that DiaPep277 and other hsp60 derived peptides and analogs, may be used to prevent, or delay the onset of T2D in patients having no apparent autoimmune component and those who are genetically predisposed to the disease.

In vivo animal models are used according to the present invention to assess the effectiveness of the hsp60 peptides and peptide analogs in T2D. These animal models utilize genetically modified strains of mice resistant to leptin (such as dp/dp or ob/ob mice), or a high fat diet model induced in otherwise healthy mice. Treatment effectiveness is measured by glucose tolerance, fasting glucose and fasting insulin levels wherein treated mice have significantly lower glucose, fasting and in the glucose tolerance test, than control animals.

Terminology and Definitions: “Functional derivatives” of the peptides of the invention as used herein covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide, do not confer toxic properties on compositions containing it and do not adversely affect the antigenic properties thereof.

These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “analog” further indicates a molecule which has the amino acid sequence according to the invention except for one or more amino acid changes. Analogs according to the present invention may comprise also peptidomimetics. “Peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with other covalent bond. A peptidomimetic according to the present invention may optionally comprises at least one bond which is an amide-replacement bond such as urea bond, carbamate bond, sulfonamide bond, hydrazine bond, or any other covalent bond. The design of appropriate “analogs” may be computer assisted.

An “effective peptide” will have the activity to achieve a desired result, such as cytokine inhibition or induction. Alternatively, an effective peptide will provide the cell with a beneficial or therapeutic effect, such as induction of release of a specific mediator. Thus reference to a particular peptide or “analog” includes the naturally occurring peptide sequence or a peptide that has the substantially the same activity as the naturally occurring sequence. “Effective peptides” of the invention also include modified peptides (with amino acid substitutions, both conservative and non-conservative) that have the same activity as a wild-type or unmodified peptide. “Salts” of the peptides of the invention contemplated by the invention are physiologically acceptable organic and inorganic salts.

As used herein and in the claims, the phrase “therapeutically effective amount” means that amount of peptide or peptide analog or composition comprising same to administer to a host to achieve the desired results for the indications disclosed herein.

The amino acids used in this invention are those which are available commercially or are available by routine synthetic methods. Certain residues may require special methods for incorporation into the peptide, and either sequential, divergent or convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” before the residue abbreviation.

Conservative substitution of amino acids as known to those skilled in the art are within the scope of the present invention. Conservative amino acid substitutions include replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

### Pharmacology

Apart from other considerations, the fact that the novel active ingredients of the invention are peptides, peptide analogs or peptidomimetics, dictates that the formulation be suitable for delivery of these type of compounds. In general, peptides are less suitable for oral administration due to susceptibility to digestion by gastric acids or intestinal enzymes, but it is now disclosed that the compositions according to the present invention are also suitable for oral administration. Other routes of administration according to the present invention are intra-articular, intravenous, intramuscular, subcutaneous, intradermal, or intrathecal.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing or liposome capturing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5, 447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of a compound effective to prevent, delay, alleviate or ameliorate symptoms of a disease of the subject being treated. Determination of a therapeutically effective amount is well within the capability of those skilled in the art.

Toxicity and therapeutic efficacy of the fragments and analogs described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

In one particularly preferred embodiment according to the present invention, the peptides are administered orally (e.g. as a syrup, capsule, or tablet).

In certain embodiments, peptide delivery can be enhanced by the use of protective excipients. This is typically accomplished either by complexing the peptide with a composition to render it resistant to acidic and enzymatic hydrolysis or by packaging the polypeptide in an appropriately resistant carrier such as a liposome. Attempts of protecting polypeptides for oral delivery have been published (e.g., U.S. Pat. Nos. 8,093,207, 7,666,446 and 7,316,819).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy, 1998, Biotechnol. Prog. 14, 108; Johnson et al., 1996, Nature Med. 2, 795; Herbert et al., 1998, Pharmaceut. Res. 15, 357) a dry powder composed of biodegradable polymeric microspheres containing the protein in a polymer matrix that can be compounded as a dry formulation with or without other agents.

The foregoing formulations and administration methods are intended to be illustrative and not limiting. It will be appreciated that, using the teaching provided herein, other suitable formulations and modes of administration can be readily devised. Formulations of the present invention suitable for oral administration may be presented as discrete units such as capsules, cachets, tablets, lozenges comprising the peptide(s) in a flavoured base, usually sucrose and acacia and tragacanth; pastilles comprising the active ingredient(s) in an inert base such as gelatin and glycerin, or sucrose and acacia; and mouth washes comprising the active ingredient(s) in a suitable liquid carrier. Each formulation generally contains a predetermined amount of the active peptide(s); as a powder or granules; or a solution or suspension in an aqueous or non-aqueous liquid such as a syrup, an elixir, an emulsion or draught and the like.

A tablet may be made by compression or moulding, optionally with one or more accessory ingredients. Compressed tablets may be prepared by compressing in a suitable machine the active peptide(s) in a free-flowing form such as a powder or granules, optionally mixed with a binder, (eg povidone, gelatin, hydroxypropylmethyl cellulose), lubricant, inert diluent, preservative, disintegrant (e.g. sodium starch glycollate, cross-linked povidone, cross-linked sodium carboxymethyl cellulose), surface active or dispersing agent. Moulded tablets may be made by moulding in a suitable machine a mixture of the powdered peptide(s) moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile.

A syrup may be made by adding the active peptide(s) to a concentrated, aqueous solution of a sugar, for example, sucrose, to which may also be added any necessary ingredients. Such accessory ingredients) may include flavourings, an agent to retard crystallisation of the sugar or an agent to increase the solubility of any other ingredients, such as a polyhydric alcohol, for example, glycerol or sorbitol.

In addition to the aforementioned ingredients, the formulations of this invention may further include one or more accessory ingredient(s) selected from diluents, buffers, flavouring agents, binders, surface active agents, thickeners, lubricants, preservatives, (including antioxidants) and the like.

According to some embodiments of the invention, the therapeutically effective amount of the hsp fragment or analog is a dosage in a range from about 0.02 mg/kg to about 10 mg/kg. Preferably, the dosage of the hsp fragment or analog according to the present invention is in a range from about 0.05 mg/kg to about 2 mg/kg, more preferably, the dosage of the hsp fragment or analog is in a range from about 0.1 mg/kg to about 1 mg/kg. It will be understood that the dosage may be an escalating dosage so that low dosage may be administered first, and subsequently higher dosages may be administered until an appropriate response is achieved. Also, the dosage of the composition can be administered to the subject in multiple administrations in the course of the treatment period in which a portion of the dosage is administered at each administration.

### Depot Systems

The parenteral route by intravenous (IV), intramuscular (IM), or subcutaneous (SC) injection is the most common and effective form of delivery for small as well as large molecular weight drugs. However, pain, discomfort and inconvenience due to needle sticks makes this mode of drug delivery the least preferred by patients. Therefore, any drug delivery technology that can at a minimum reduce the total number of injections is preferred. Such reductions in frequency of drug dosing in practice may be achieved through the use of injectable depot formulations that are capable of releasing drugs in a slow but predictable manner and consequently improve compliance. For most drugs, depending on the dose, it may be possible to reduce the injection frequency from daily to once or twice monthly or even longer (6 months). In addition to improving patient comfort, less frequent injections of drugs in the form of depot formulations smoothes out the plasma concentration-time profile by eliminating the hills and valleys. Such smoothing out of plasma profiles has the potential to not only boost the therapeutic benefit in most cases, but also to reduce any unwanted events, such as immunogenicity etc. often associated with large molecular weight drugs.

Microparticles, implants and gels are the most common forms of biodegradable polymeric devices used in practice for prolonging the release of drugs in the body. Microparticles are suspended in an aqueous media right before injection and one can load as much as 40% solids in suspensions. Implant/rod formulations are delivered to SC/IM tissue with the aid of special needles in the dry state without the need for an aqueous media. This feature of rods/implants allows for higher masses of formulation, as well as drug content to be delivered. Further, in the rods/implants, the initial burst problems are minimized due to much smaller area in implants compared to the microparticles. Besides biodegradable systems, there are non-biodegradable implants and infusion pumps that can be worn outside the body. Non-biodegradable implants require a doctor's visit not only for implanting the device into the SC/IM tissue but also to remove them after the drug release period.

Injectable compositions containing microparticle preparations are particularly susceptible to problems. Microparticle suspensions may contain as much as 40% solids as compared with 0.5-5% solids in other types of injectable suspensions. Further, microparticles used in injectable depot products, range in size up to about 250μm (average, 60-100 μm), as compared with a particle size of less than 5μm recommended for IM or SC administration. The higher concentrations of solids, as well as the larger solid particle size require larger size of needle (around 18-21 gauge) for injection. Overall, despite the infrequent uses of larger and uncomfortable needles, patients still prefer less frequently administered dosage forms over everyday drug injections with a smaller needle.

Biodegradable polyesters of poly(lactic acid) (PLA) and copolymers of lactide and glycolide referred to as poly(lactide-co-glycolide) (PLGA) are the most common polymers used in biodegradable dosage forms. PLA is hydrophobic molecule and PLGA degrades faster than PLA because of the presence of more hydrophilic glycolide groups. These biocompatible polymers undergo random, non-enzymatic, hydrolytic cleavage of the ester linkages to form lactic acid and glycolic acid, which are normal metabolic compounds in the body. Resorbable sutures, clips and implants are the earliest applications of these polymers. Southern Research Institute developed the first synthetic, resorbable suture (Dexon®) in 1970. The first patent describing the use of PLGA polymers in a sustained release dosage form appeared in 1973 (U.S. Pat. No. 3,773,919).

Today, PLGA polymers are commercially available from multiple suppliers; Alkermes (Medisorb polymers), Absorbable Polymers International [formerly Birmingham Polymers (a Division of Durect)], Purac and Boehringer Ingelheim. Besides PLGA and PLA, natural cellulosic polymers such as starch, starch derivatives, dextran and non-PLGA synthetic polymers are also being explored as biodegradable polymers in such systems.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the amended claims

### EXAMPLES

### Example 1

### Experimental Animal Models for T2D

Mice genetically prone to T2D (dp/dp or ob/ob strains, resistant to leptin), and/or healthy C57BL/6 mice induced to develop T2D by a high fat diet are used in vivo to assess the activity of hsp60 peptides and analogs in protecting against or treatment of T2D. The db/db model is described, for example in Dray et al., Am. J. Physiol. Endocrinol Metab 2010, 298, E1161-E1169. In the fat diet model, the diet (for example TD.0811 of Harlan™, or D12492 of Research Diets Inc. containing 21% and 34.9% fat respectively) is initiated after weaning and the mice express T2D by 10 weeks of age. These, as well as other animal models for T2D were reviewed by Srinivasan and Ramarao (Indian J Med Res 2007, 125, 451-472).

In a typical trial, groups of T2D mice (10 mice per group) are treated early in the course of the disease (about 7-10 weeks of age). Negative control mice consist of healthy mice and positive control mice are T2D treated with placebo.

Treatment with DiaPep277 or other hsp60 peptides or analogs, is performed once or several times a week with doses of 100-500 micrograms injected subcutaneously (with adjuvant such as intralipid) or IP, IM or IV (without adjuvant). Glucose tolerance, fasting glucose and fasting insulin levels are measured 1-2 months after onset of treatment.

In one specific in-vivo trial, 70 male C57BL/N mice were weighed, measured for blood glucose levels and divided into seven groups, 10 mice/group as following:

1. Regular diet, no treatment;

2. High fat (60%) diet;

3. High fat (60%) diet+intralipid SC once a week;

4. High fat (60%) diet+100 μg DiaPep277 in intralipid SC, once per treatment;

5. High fat (60%) diet+200 μg DiaPep277 in intralipid SC, once a week;

6. High fat (60%) diet+500 μg DiaPep277 in intralipid SC, once a week;

7. High fat (60%) diet+100 μg DiaPep277 in PBS IP, three times per week.

Animals of groups 2-7 were fed with 60% fat diet of from age of 5 weeks until termination of the trial. Fasting glucose levels were tested once a week. After about 16 weeks the trial was terminated and Hemoglobin A1C and C-peptide levels were measured using methods known in the art.

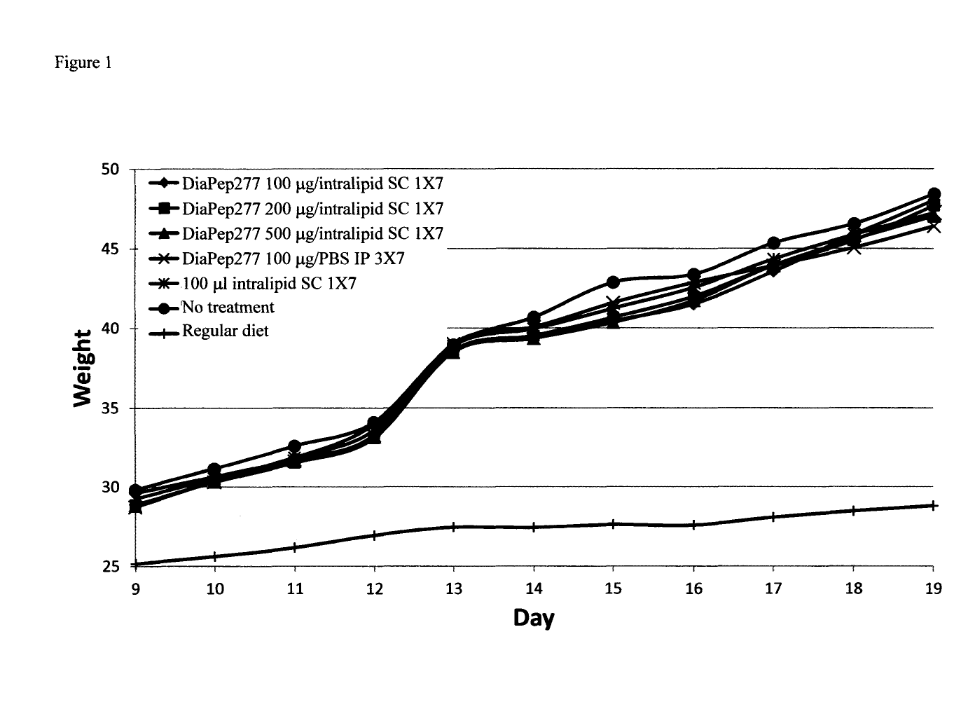

It was demonstrated that, while animal appetite and weight did not change in response to any of the DiaPep277 treatments (FIG. 1), blood fasting glucose levels were decreased in response to some of the treatment regimens. The decrease in blood glucose levels was particularly significant (p=0.0178) in the group treated with 100 μg DiaPep277 in PBS IP, three times per week, versus the untreated group.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1-36. (canceled)

37. A method of treating or alleviating Type 2 diabetes (T2D) comprising administering to a subject in need thereof, a composition comprising a peptide derived from hsp60 or a peptide analog thereof.

38. The method of claim 37 wherein the peptide analog comprises a sequence corresponding to amino acid residues 437-460 of human hsp60 having the sequence: Val-Leu-Gly-Gly-Gly-X₁-Ala-Leu-Leu-Arg-X₂-IIe-Pro-Ala-Leu-Asp-Ser-Leu-X₃-Pro-Ala-Asn-Glu-Asp (SEQ ID NO:1), wherein X₁ is a Cys or Val residue, X₂ is a Cys or Val residue, and X₃ is a Thr or Lys residue.

39. The method of claim 38 wherein the peptide analog is a Val⁶, Val¹¹ analog of residues 437-460 of hsp60, as set forth in SEQ ID NO:2:
(SEQ ID NO: 2)
1 6 11
Val-Leu-Gly-Gly-Gly-Val-Ala-Leu-Leu-Arg-Val-Ile-Pro-Ala-Leu-Asp-Ser-Leu-Thr-Pro-
24
Ala-Asn-Glu-Asp,
herein denoted DiaPep277.

40. The method of claim 37 wherein the hsp60 fragment peptide is selected from the group consisting of:
residues 31-50 of human hsp60:
(SEQ ID NO: 3)
Lys-Phe-Gly-Ala-Asp-Ala-Arg-Ala-Leu-Met-Leu-Gln-
Gly-Val-Asp-Leu-Leu-Ala-Asp-Ala;
residues 136-155 of human hsp60:
(SEQ ID NO: 4)
Asn-Pro-Val-Glu-Ile-Arg-Arg-Gly-Val-Met-Leu-Ala-
Val-Asp-Ala-Val-Ile-Ala-Glu-Leu;
residues 151-170 of human hsp60:
(SEQ ID NO: 5)
Val-Ile-Ala-Glu-Leu-Lys-Lys-Gln-Ser-Lys-Pro-Val-
Thr-Thr-Pro-Glu-Glu-Ile-Ala-Gln;
residues 166-185 of human hsp60:
(SEQ ID NO: 6)
Glu-Glu-Ile-Ala-Gln-Val-Ala-Thr-Ile-Ser-Ala-Asn-
Gly-Asp-Lys-Glu-Ile-Gly-Asn-Ile;
residues 195-214 of human hsp60:
(SEQ ID NO: 7)
Arg-Lys-Gly-Val-Ile-Thr-Val-Lys-Asp-Gly-Lys-Thr-
Leu-Asn-Asp-Glu-Leu-Glu-Ile-Ile;
residues 255-274 of human hsp60:
(SEQ ID NO: 8)
Gln-Ser-Ile-Val-Pro-Ala-Leu-Glu-Ile-Ala-Asn-Ala-
His-Arg-Lys-Pro-Leu-Val-Ile-Ile;
residues 286-305 of human hsp60:
(SEQ ID NO: 9)
Leu-Val-Leu-Asn-Arg-Leu-Lys-Val-Gly-Leu-Gln-Val-
Val-Ala-Val-Lys-Ala-Pro-Gly-Phe;
residues 346-365 of human hsp60:
(SEQ ID NO: 10)
Gly-Glu-Val-Ile-Val-Thr-Lys-Asp-Asp-Ala-Met-Leu-
Leu-Lys-Gly-Lys-Gly-Asp-Lys-Ala;
residues 421-440 of human hsp60:
(SEQ ID NO: 11)
Val-Thr-Asp-Ala-Leu-Asn-Ala-Thr-Arg-Ala-Ala-Val-
Glu-Glu-Gly-Ile-Val-Leu-Gly-Gly;
residues 436-455 of human hsp60:
(SEQ ID NO: 12)
Ile-Val-Leu-Gly-Gly-Gly-Cys-Ala-Leu-Leu-Arg-Cys-
Ile-Pro-Ala-Leu-Asp-Ser-Leu-Thr;
residues 466-485 of human hsp60:
(SEQ ID NO: 13)
Glu-Ile-Ile-Lys-Arg-Thr-Leu-Lys-Ile-Pro-Ala-Met-
Thr-Ile-Ala-Lys-Asn-Ala-Gly-Val;
residues 511-530 of human hsp60:
(SEQ ID NO: 14)
Val-Asn-Met-Val-Glu-Lys-Gly-Ile-Ile-Asp-Pro-Thr-
Lys-Val-Val-Arg-Thr-Ala-Leu-Leu;
residues 343-366 of human hsp60:
(SEQ ID NO: 15)
Gly-Lys-Val-Gly-Glu-Val-Ile-Val-Thr-Lys-Asp-Asp-
Ala-Met.

41. The method according to claim 37 wherein administration is via a route selected from the group consisting of: intramuscular, intravenous, oral, intraperitoneal, subcutaneous, topical, intradermal or transdermal delivery.

42. The method according to claim 37 wherein the composition comprises at least 2 mg of the hsp60 derived peptide or peptide analog.

43. The method according to claim 42 wherein the composition comprises 2-50 mg of the hsp60 derived peptide or peptide analog.

44. The method according to claim 37 wherein the hsp60 derived peptide or peptide analog is administered to a subject in need thereof in a schedule selected from: 2-24 times per month and 2-5 times per week.

45. The method according to claim 37 wherein a composition comprising 2-10 mg of the hsp60 derived peptide or peptide analog is administered at least once a week by a route selected from the group consisting of: subcutaneous injection, intra-peritoneal (IP) injection, intra-muscular (IM) injection and intra-venous (IV) injection.

46. The method according to claim 37 wherein a composition comprising 50-500 mg hsp60 peptide or analog is administered orally 1-4 times per month.

47. The method according to claim 37 wherein the hsp60 derived peptide or peptide analog is administered as part of a treatment regimen comprising administering to the patient insulin.

48. The method according to claim 37 wherein the subject does not receive insulin as part of its treatment.

49. The method of claim 37 for suppression, prevention or treatment of at least one complication of T2D.

50. The method according to claim 49 wherein the complication of T2D is selected from the group consisting of: metabolic syndrome, fatty liver, neuropathy, nephropathy, heart disease, peripheral vascular disease and cancer.

51. The method according to claim 49 wherein the method further comprises administration of at least one additional anti diabetic agents.

52. A method of delaying the onset of T2D in patients having no apparent autoimmune component and those who are genetically predisposed to the disease, comprising administering of an hsp60 derived peptide or peptide analog.

53. The method according to claim 52 wherein the hsp60 peptide analog is DiaPep277 (SEQ ID NO: 2).

54. A long acting pharmaceutical composition comprising DiaPep277 or a pharmaceutically acceptable salt thereof, specifically formulated for providing a therapeutically effective amount of the peptide over a period selected from 2-6 days, one week, two weeks or longer.

55. A method of treatment of T2D comprising administering to a subject in need thereof the long acting pharmaceutical composition of claim 54.

56. The long acting pharmaceutical composition of claim 54 in depot form suitable for injection or implantation at a medically acceptable location in a subject in need thereof.
